(19) United States
(12) Patent Application Publication
(10) Pub. No.: US 2023/0374141 A1
(43) Pub. Date: Nov. 23, 2023

(54) COMBINATION THERAPY FOR ALPORT RENAL DISEASE

(71) Applicant: Father Flanagan's Boys' Home Doing Business as Boys Town National Research Hospital, Omaha, NE (US)

(72) Inventor: Dominic Cosgrove, Omaha, NE (US)

(21) Appl. No.: 18/248,003

(22) PCT Filed: Oct. 5, 2021
(86) PCT No.: PCT/US2021/053624

§ 371 Date: Apr. 5, 2023

Related U.S. Application Data
(60) Provisional application No. 63/087,560, filed on Oct. 5, 2020.

(51) Int. Cl. C07K 16/28; A61K 31/403; A61K 31/401; A61K 45/06; A61K 38/17; A61P 13/12

(52) U.S. Cl. CPC C07K 16/2842; A61K 31/403; A61K 31/401; A61K 45/06; A61K 38/1703; A61P 13/12; A61K 2039/505

(57) ABSTRACT

The present disclosure relates to a combination therapy for the treatment of Alport renal disease. In particular, to the treatment of Alport renal disease by administration of both an α1 integrin blocking agent and either an angiotensin-converting enzyme (ACE) inhibitor or an angiotensin-receptor blocker (ARB). Preliminary studies have shown the combination therapy as described herein elongates the onset of end-stage renal disease and other symptoms of Alport renal disease.

### CROSS-REFERENCE

This application is a U.S. National Phase of International Patent Application No. PCT PCT/US2021/053624, filed Oct. 5, 2021, which claims priority to provisional application U.S. Ser. No. 63/087,560, filed Oct. 5, 2020, which is incorporated herein by reference in its entirety.

### TECHNICAL FIELD

The present disclosure relates to a combination therapy for the treatment of Alport renal disease. In particular, to the treatment of Alport renal disease by administration of both an α1 integrin blocking agent and either an angiotensin-converting enzyme (ACE) inhibitor or an angiotensin-receptor blocker (ARB).

### TECHNICAL BACKGROUND

Alport syndrome is a genetic disorder characterized by abnormalities in the basement membranes of the glomerulus (leading to hematuria, glomerulosclerosis, and end-stage kidney disease (ESRD)), cochlea (causing deafness), and eye (resulting in lenticonus and perimacular flecks). Alport syndrome is a primary basement membrane disorder caused by mutations in the collagen type IV COL4A3, COL4A4, or COL4A5 genes. Mutations in any of these genes prevent the proper production or assembly of the type IV collagen network, which is an important structural component of basement membranes in the renal glomerulus, inner ear, and eye. Basement membranes are thin, sheet-like structures that separate and support cells in many tissues. The abnormalities of type IV collagen in kidney glomerular basement membranes leads to irregular thickening, resulting in thinning and splitting of these basement membranes, causing gradual scarring (fibrosis) of the kidneys. Alport Syndrome has a delayed onset and causes progressive kidney damage. The glomeruli and other normal kidney structures such as tubules are gradually replaced by scar tissue, gradually reducing glomerular filtration rates leading to kidney failure. Hearing loss and an abnormality in the shape of the lens called anterior lenticonus are other important features of Alport Syndrome. People with anterior lenticonus may have problems with their vision and may develop cataracts. The prevalence of Alport syndrome is estimated at approximately 1 in 5,000 births and it is estimated that the syndrome accounts for approximately 2.1 percent of pediatric patients with ESRD.

Currently there is no specific treatment for Alport Syndrome; treatments are symptomatic. Patients are advised on how to manage the complications of kidney failure and the proteinuria that develops is often treated off label with ACE inhibitors. Once kidney failure has developed, patients are given dialysis or can benefit from a kidney transplant, although this can cause problems. The body may reject the new kidney as it contains normal type IV collagen, which may be recognized as foreign by the immune system.

While several therapeutics are moving to human clinical trials for the treatment of Alport renal disease, none have been shown to provide significant benefit over the current standard of care, which is treatment with angiotensin converting enzyme (ACE) blockers such as ramipril or with angiotension receptor blockers (ARBs). Thus, there is a need for improved methods for the treatment of Alport renal disease.

### SUMMARY OF PREFERRED EMBODIMENTS

The present disclosure includes a method of treating Alport syndrome in a subject, the method including administering both an α1 integrin blocking agent and either an angiotensin-converting enzyme (ACE) inhibitor or an angiotensin-receptor blocker (ARB) to the subject.

The present disclosure includes a method of preventing glomerular disease progression in a subject diagnosed with Alport syndrome, the method including administering both an α1 integrin blocking agent and either an angiotensin-converting enzyme (ACE) inhibitor or an angiotensin-receptor blocker (ARB) to the subject.

The present disclosure includes a method of treating glomerulonephritis in a subject, the method including administering both an α1 integrin blocking agent and either an angiotensin-converting enzyme (ACE) inhibitor or an angiotensin-receptor blocker (ARB) to the subject.

The present disclosure includes a method of treating glomerular injury due to biomechanical strain in Alport syndrome, the method including administering both an α1 integrin blocking agent and either an angiotensin-converting enzyme (ACE) inhibitor or an angiotensin-receptor blocker (ARB) to the subject.

The present disclosure includes a method of inhibiting deposition of laminin 211 in the glomerular basement membrane (GBM) in a subject, the method including administering an α1 integrin blocking agent and either an angiotensin-converting enzyme (ACE) inhibitor or an angiotensin-receptor blocker (ARB) to the subject.

The present disclosure includes a method of inhibiting mesangial cell process invasion of the glomerular capillary loop in a kidney of a subject, the method including administering an α1 integrin blocking agent and either an angiotensin-converting enzyme (ACE) inhibitor or an angiotensin-receptor blocker (ARB) to the subject.

The present disclosure includes a method of inhibiting Alport glomerular pathogenesis in a subject; the method including: determining that the subject is at risk for developing Alport glomerular disease; and administering both an α1 integrin blocking agent and either an angiotensin-converting enzyme (ACE) inhibitor or an angiotensin-receptor blocker (ARB) to the subject. In some aspects, of the method, the determination that the subject is at risk for developing Alport glomerular disease is determined by family medical history, genetic testing, immunodiagnostic skin biopsy testing, and/or molecular diagnostic marker testing. In some aspects, the determination that the subject is at risk for developing Alport glomerular disease is made prior to the onset of proteinuria in the subject.

With the methods of the present disclosure, one or more sensory and/or hearing losses associated with Alport syndrome is delayed and/or treated.

With the methods of the present disclosure, the al integrin blocking agent includes an α1 integrin neutralizing antibody.

With the methods of the present disclosure, the al integrin blocking agent includes a small molecule inhibitor. In some aspects, small molecule inhibitor is obtustatin.

With the methods of the present disclosure, the al integrin blocking agent may prevent signaling through the α1β1 integrin receptor.

With the methods of the present disclosure, the ACE inhibitor is selected from benazepril, captopril, enalapril, fosinopril, lisinopril, moexipril, perindopril, quinapril, ramipril, and/or trandolapril. In some aspects, the ACE inhibitor is selected from ramipril and/or analapril.

With the methods of the present disclosure, the ARB is selected from candesartan, irbesartan, olmesartan, losartan, valsartan, telmisartan, and/or eprosartan.

With the methods of the present disclosure, the administration of both an α1 integrin blocking agent and an ACE inhibitor or an ARB is synergistic.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. Proteinuria is markedly reduced in α1 null Alport mice treated with ramipril compared with untreated al-null Alport mice. Urine was collected from the indicated treatment groups at the indicated ages. Total protein was measured by Bradford assay and normalized to urinary creatinine.

FIG. 2. shows a chart demonstrating the survival rate (in weeks) of mice suffering with Alport syndrome. The age of death is indicated in weeks. The chart compares mice with Alport syndrome that are untreated (i.e., receiving no medicine to treat the Alport syndrome), mice treated with Ramipril (an ACE inhibitor), DKO mice left untreated (i.e., mice that did not receive Ramipril), and a combination therapy, i.e., DKO mice treated with Ramipril.

FIGS. 3A-3F are color dual immunofluorescence confocal images of glomeruli stained with antibodies against laminin α5 (a GBM marker) and laminin α2. FIGS. 3A-3C are untreated DKO mice. FIGS. 3D-3F are DKO mice treated with Ramipril. FIGS. 3A and 3D show laminin α5 data. FIGS. 3B and 3E show laminin α2 data. FIGS. 3C and 3F show a merge of the laminin α5 and laminin α2 data. These figures demonstrate that laminin α2 is present in the GBM of DKO mice but not ramipril-treated DKO mice at 10 weeks of age.

FIGS. 4A-4F are color dual immunofluorescence confocal images of glomeruli stained with antibodies against nidogen (a GBM marker) and collagen III. FIGS. 4A-4C are untreated DKO mice. FIGS. 4D-4F are DKO mice treated with Ramipril. FIGS. 4A and 4D show nidogen data. FIGS. 4B and 4E show collagen 3 α1 data. FIGS. 3C and 3F show a merge of the nidogen and collagen 3 α1 data. These figures demonstrate that collagen III is present in the GBM of DKO mice but not ramipril-treated DKO mice at 10 weeks of age.

FIG. 5 is a color dual immunofluorescence SR-SIM analysis using antibodies against collagen III (in green) and α-actinin 4 (in red) demonstrate that the collagen III in the GBM of Alport mice appears in contact with podocyte foot processes. The arrowheads denote areas where clear contact of collagen III and podocyte foot processes is evident. This figure demonstrates that collagen III in the GBM of Alport mice is proximal to the podocyte foot processes and thus may activate collagen receptors.

FIGS. 6A-6F are color dual immunofluorescence confocal images of glomeruli stained with antibodies against laminin α5 (a GBM marker) and laminin α2. FIGS. 6A-6C were taken at 20 weeks. FIGS. 6D-6F were taken at 25 weeks. FIGS. 6A and 6D show laminin α5 data. FIGS. 6B and 6E show laminin α2 data. FIGS. 6C and 6F show a merge of the laminin α5 and laminin α2 data. These figures show that Laminin 211 is deposited in the GBM in ramipril-treated DKO mice after 20 weeks and at or before 25 weeks of age.

FIG. 7 shows a color chart comparing glomerular filtration rates via the normalized luminosity over time of the Ramipril-treated DKO mice at age 20-weeks and 25-weeks. The red data is representative of the 20-week old mice and the blue data is representative of the 25-week old mice. FIG. 7 show that GFR is reduced in DKO ramipril mice at 25 versus 20 weeks of age.

FIGS. 8A-8D show assay data in color demonstrating that α1β1 integrin functions along the CDC42 activation axis in cultured mesangial cells. FIG. 8A shows Boyden chamber cell migration in response to fetal calf serum. FIG. 8B shows stimulation of filopodial formation by ET-1, which is indicated by the arrows. FIG. 8C shows color data from an ELISA assay for activated CDC42 following stimulation of mesangial cells with LPS. FIG. 8D shows Activation of CDC42 by LPS as demonstrated using pull-down assay with antibodies against activated CDC42. These figures demonstrate that al-null mesangial cells show reduced migration, failure to form filopodia in response to ET-1 stimulation, and failure to activate CDC42 in response to LPS.

Various embodiments of the treatment methods disclosure herein will be described in detail with reference to the figures. Reference to various embodiments does not limit the scope of the inventions. Figures represented herein are not limitations to the various embodiments according to the invention and are presented for exemplary illustration of the inventions.

### DETAILED DESCRIPTION

In a preferred embodiment, treatments and methods of treating Alport syndrome are provided which comprise combining both (1) an α1 integrin blocking agent and (2) either an angiotensin-converting enzyme (ACE) inhibitor or an angiotensin-receptor blocker (ARB). Another preferred embodiment is a method of comprising administering to a subject diagnosed with Alport syndrome both (1) an α1 integrin blocking agent and (2) either an ACE inhibitor or an ARB. Still another preferred embodiment is a method of treating glomerulonephritis comprising administering to a subject both (1) an α1 integrin blocking agent and (2) either an ACE inhibitor or an ARB. Yet another preferred embodiment disclosed herein is a method of preventing glomerular disease progression in a subject diagnosed with Alport syndrome, the method comprising administering to the subject diagnosed with Alport syndrome both (1) an α1 integrin blocking agent and (2) either an ACE inhibitor or an ARB. Still another preferred embodiment is a method of treating glomerular injury due to biomechanical strain in Alport syndrome, the method comprising administering to a subject diagnosed with Alport syndrome both (1) an α1 integrin blocking agent and (2) either an ACE inhibitor or an ARB. Another preferred embodiment is a method of inhibiting deposition of laminin 211 in the glomerular basement membrane (GBM), the method comprising administering to a subject diagnosed with Alport syndrome both (1) an α1 integrin blocking agent and (2) either an ACE inhibitor or an ARB. A further preferred embodiment is a method of inhibiting mesangial cell process invasion of the glomerular capillary loop in a kidney of a subject, the method comprising administering to the subject diagnosed with Alport syndrome both (1) an α1 integrin blocking agent and (2) either an ACE inhibitor or an ARB. Yet a further preferred embodiment comprises a method of inhibiting Alport glomerular pathogenesis in a subject, the method comprising administering to the subject diagnosed with Alport syndrome both (1) an α1 integrin blocking agent and (2) either an ACE inhibitor or an ARB.

The embodiments described herein are not limited to particular ACE inhibitor or an ARB tested or disclosed herein, but rather the methods disclosed herein may be applied in combination with other ACE inhibitors or ARBs whether in clinical testing now or not. The foregoing preferred embodiments, and other embodiments, disclosed herein, provide may unexpected benefits relating to the treatment of Alport syndrome and the treatment of symptoms of Alport syndrome. One such benefit is a synergistic improvement in the treatment of Alport disease in a subject. One such benefit is to provide a synergistic slowing of the progression of the syndrome and its effects on the subject. Still another benefit is to extend the life-expectancy of a subject having Alport syndrome. These and other benefits of the present disclosure will be made clear in the following disclosure.

### Definitions

For clarity, certain terms are first defined. Unless defined otherwise, all technical and scientific terms used herein have the same meaning as commonly understood in the field. Many methods and materials similar, modified, or equivalent to those described herein can be used in the practice of the embodiments described herein without undue experimentation, the preferred materials and methods are described herein. In describing and claiming the embodiments described herein, the following terminology will be used in accordance with the definitions set out below.

It is further to be understood that all terminology used herein is for the purpose of describing particular embodiments only and is not intended to be limiting in any manner or scope. For example, as used in this specification and the appended claims, the singular forms “a,” “an” and “the” can include plural referents unless the content clearly indicates otherwise. Thus, unless otherwise specified, “a,” “an,” “the,” and “at least one” are used interchangeably and mean one or more than one.

It should also be noted that the term “or” is generally employed in its sense including “and/or” unless the content clearly dictates otherwise. The term “and/or” means one or all of the listed elements or a combination of any two or more of the listed elements. Further, all units, prefixes, and symbols may be denoted in its SI accepted form.

The terms “comprises,” and variations thereof, do not have a limiting meaning where these terms appear in the description and claims.

As used herein “in vitro” is in cell culture and “in vivo” is within the body of a subject.

As used herein, “isolated” refers to material that has been either removed from its natural environment (e.g., the natural environment if it is naturally occurring), produced using recombinant techniques, or chemically or enzymatically synthesized, and thus is altered “by the hand of man” from its natural state.

The words “preferred” and “preferably” refer to embodiments of the inventions that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the inventions.

As used herein, the term “subject” includes, but is not limited to, humans and non-human vertebrates. In preferred embodiments, a subject is a mammal, particularly a human. A subject may be an individual. A subject may be an “individual,” “patient,” or “host. In some aspects, a subject is an individual diagnosed with Alport syndrome. Diagnosis may be by any of a variety of means, including, but not limited to, family history, clinical presentation, pathological determination, and/or genetic testing. Such as subject may be a male or a female. Non-human vertebrates include livestock animals, companion animals, and laboratory animals. Non-human subjects also include non-human primates as well as rodents, such as, but not limited to, a rat or a mouse. Non-human subjects also include, without limitation, chickens, horses, cows, pigs, goats, dogs, cats, guinea pigs, hamsters, mink, and rabbits.

As used herein “treating” or “treatment” can include therapeutic and/or prophylactic treatments. Desirable effects of treatment include preventing occurrence or recurrence of disease, alleviation of symptoms, diminishment of any direct or indirect pathological consequences of the disease, decreasing the rate of disease progression, amelioration or palliation of the disease state, and remission or improved prognosis.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present inventions. At the very least, and not as an attempt to limit the doctrine of equivalents to the scope of the claims, each numerical parameter should at least be construed in light of the number of reported significant digits and by applying ordinary rounding techniques.

Numeric ranges recited within the specification are inclusive of the numbers defining the range and include each integer within the defined range. Throughout this disclosure, various aspects of the preferred embodiments are presented in a range format. It should be understood that the description in range format is merely for convenience and brevity and should not be construed as an inflexible limitation on the scope of the invention. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾. This applies regardless of the breadth of the range.

Notwithstanding that the numerical ranges and parameters setting forth the broad scope of the inventions are approximations, the numerical values set forth in the specific examples are reported as precisely as possible. All numerical values, however, inherently contain a range necessarily resulting from the standard deviation found in their respective testing measurements.

For any method disclosed herein that includes discrete steps, the steps may be conducted in any feasible order. And, as appropriate, any combination of two or more steps may be conducted simultaneously.

In several places throughout the application, guidance is provided through lists of examples, which examples can be used in various combinations. In each instance, the recited list serves only as a representative group and should not be interpreted as an exclusive list. It is to be understood that the particular examples, materials, amounts, and procedures are to be interpreted broadly in accordance with the scope and spirit of the inventions as set forth herein.

All headings are for the convenience of the reader and should not be used to limit the meaning of the text that follows the heading, unless so specified.

### Combination Therapy for Alport Syndrome

The present disclosure provides new combination therapies for the treatment of Alport syndrome. Alport syndrome (incidence about 1 in 5000) is characterized by delayed onset progressive glomerulonephritis associated with sensorineural hearing loss and retinal flecks (Kashtan and Michael, 1996, Kidney Int; 50(5):1445-1463). The most common form (80%) is X-linked and caused by mutations in the type IV collagen COL4A5 gene (Barker et al., 1990, Science; 248(4960):1224-7). The two autosomal forms of the disease account for the remaining 20% of Alport patients and result from mutations in the COL4A3 and COL4A4 genes (Mochizuki et al., 1994, Nat Genet; 8(1):77-81). The α3(IV), α4(IV) and α5(IV) proteins form a heterotrimer and are assembled into a subepithelial network in the mature glomerular basement membrane (GBM) that is physically and biochemically distinct from a subendothelial type IV collagen network comprised of α1(IV) and α2(IV) heterotrimers (Kleppel et al., 1992, J Biol Chem; 267(6):4137-4142). Mutation in any one of the three type IV collagen genes that cause Alport syndrome results in the absence of all three proteins in the glomerular basement membrane (GBM) due to an obligatory association in basement membrane collagen assembly to form functional heterotrimers (Kalluri and Cosgrove, 2000, J Biol Chem; 275(17):12719-12724). Thus, the net result for all genetic forms of Alport syndrome is the absence of the α3(IV) α4(IV) α5(IV) subepithelial collagen network, resulting in a thinner GBM type IV collagen network comprised only of α1(IV) and α2(IV) heterotrimers.

The current standard of care for the treatment of Alport renal disease is treatment with an ACE inhibitor, typically ramipril, or an ARB. While several therapeutics are moving to human clinical trials, none have been shown to provide significant improvement of renal function and lifespan over ACE inhibitors or ARBs.

The present disclosure provides methods for the treatment of Alport renal disease, resulting in improved renal function, slowed progression of proteinuria, and increased lifespan. The methods disclosed herein provide for a combination therapy with the dual administration of both an α1 integrin blocking agent and either an ACE inhibitor or an ARB. This dual therapy prevents mesangial filopodial invasion and the deposition of mesangial proteins in the GBM of Alport patients.

This dual therapy shows significant synergistic benefit when compared to treatment with an ACE inhibitor alone, which is the current standard of care for Alport renal disease. As shown in Example 1, administration of an ACE inhibitor such as ramipril, in combination with al integrin blockade provides a significant synergistic improvement of renal health and lifespan compared to treatment with an ACE inhibitor alone. The tripling of lifespan in the Alport mouse model seen in Example 1 is unprecedented and if translated to humans could portend survival to 60-70 years of age for Alport patients with severe mutations.

The present disclosure includes methods of treating Alport syndrome in a subject by administering both 1) an α1 integrin blocking agent and 2) either an angiotensin-converting enzyme (ACE) inhibitor or an angiotensin-receptor blocker (ARB) to the subject.

The present disclosure includes methods of preventing glomerular disease progression in a subject diagnosed with Alport syndrome by administering both 1) an α1 integrin blocking agent and 2) either an ACE inhibitor or ARB to the subject.

The present disclosure includes methods of treating glomerulonephritis in a subject by administering both 1) an α1 integrin blocking agent and 2) either an angiotensin-converting enzyme inhibitor or an angiotensin-receptor blocker to the subject.

The present disclosure includes methods of treating kidney injury due to biomechanical strain in Alport syndrome by administering both 1) an α1 integrin blocking agent and 2) either an angiotensin-converting enzyme (ACE) inhibitor or an angiotensin-receptor blocker (ARB) to the subject.

The present disclosure includes methods of inhibiting deposition of laminin 211 in the glomerular basement membrane (GBM) in a subject by administering both 1) an α1 integrin blocking agent and 2) either an angiotensin-converting enzyme (ACE) inhibitor or an angiotensin-receptor blocker (ARB) to the subject.

The present disclosure includes methods of inhibiting mesangial cell process invasion of the glomerular capillary loop in a kidney of a subject by administering both 1) an α1 integrin blocking agent and 2) either an angiotensin-converting enzyme (ACE) inhibitor or an angiotensin-receptor blocker (ARB) to the subject.

The present disclosure includes methods of inhibiting Alport glomerular pathogenesis in a subject by determining that the subject is at risk for developing Alport glomerular disease and administering both 1) an α1 integrin blocking agent and 2) either an angiotensin-converting enzyme (ACE) inhibitor or an angiotensin-receptor blocker (ARB) to the subject. The determination that the subject is at risk for developing Alport glomerular disease may be determined, for example, by family medical history, genetic testing, immunodiagnostic skin biopsy testing, and/or molecular diagnostic marker testing. In some applications, a determination that the subject is at risk for developing Alport glomerular disease may be made prior to the onset of proteinuria in the subject.

The methods described herein may, for example, inhibit migration of mesangial cells, inhibit irregular deposition of mesangial laminin 211 in the GBM, inhibit invasion of the capillary loops by mesangial cell processes, inhibit mesangial filopodial invasion of the glomerular capillary tuft, and/or prevent, or slow the onset and/or progression of proteinuria. With the methods described herein, one or more sensory and/or hearing losses associated with Alport syndrome may also be delayed, treated, and/or prevented.

α1 integrin blocking agents include, but are not limited to, antibodies that bind to al integrin. In some aspects, such an antibody inhibits, blocks, and/or neutralizes one or more functions of al integrin. A variety of such antibodies are known or can be produced and characterized by any of a variety of means known to the skilled artisan. See, for example, de Fougerolles et al., 2000, J Cin Invest; 105(6):721-729; Fiorucci et al., 2002, Immunity; 17(6):769-780 (doi: 10.1016/s1074-7613(02)00476-4); and Conrad et al., 2007, Nature Medicine; 13(7):836-842 (doi: 10.1038/nm1605).

As will be understood by those in the art, the term “antibody” extend to all antibodies from all species, and antigen binding fragments thereof, including dimeric, trimeric and multimeric antibodies; bispecific antibodies; chimeric antibodies; human and humanized antibodies; recombinant and engineered antibodies, and fragments thereof. The term “antibody” is thus used to refer to any antibody-like molecule that has an antigen binding region, and this term includes antibody fragments such as, for example, Fab′, Fab, F(ab′)2, single domain antibodies (DABs), Fv, scFv (single chain Fv), linear antibodies, diabodies, and the like. The techniques for preparing and using various antibody-based constructs and fragments are well known in the art.

In certain embodiments, the antibodies employed may be “humanized” antibodies. Humanized” antibodies are generally chimeric monoclonal antibodies from mouse, rat, or other non-human species, bearing human constant and/or variable region domains. Various humanized monoclonal antibodies for use in the present disclosure will be chimeric antibodies wherein at least a first antigen binding region, or complementarity determining region (CDR), of a mouse, rat or other non-human monoclonal antibody is operatively attached to, or “grafted” onto, a human antibody constant region or “framework.” Humanized monoclonal antibodies for use herein may also be monoclonal antibodies from non-human species wherein one or more selected amino acids have been exchanged for amino acids more commonly observed in human antibodies. This can be readily achieved through the use of routine recombinant technology, particularly site-specific mutagenesis.

Entirely human antibodies may also be prepared and used in the present disclosure. Such human antibodies may be obtained from healthy subjects by simply obtaining a population of mixed peripheral blood lymphocytes from a human subject, including antigen-presenting and antibody-producing cells, and stimulating the cell population in vitro.

α1 integrin blocking agents include, but are not limited to, small molecule inhibitors of al integrin. Small molecule inhibitors of al integrin include, but are not limited to, the small molecule inhibitor obtustatin. Obtustatin, a novel disintegrin purified from the venom of the Vipera lebetina obtusa viper, is a potent and selective inhibitor of α1β1 integrin (Marcinkiewicz et al., 2003, Cancer Res; 63(9):2020-3).

Angiotensin-converting enzyme inhibitors (ACE inhibitors) are a group of medicines used to treat certain heart and kidney conditions. They block the production of angiotensin II, a substance that narrows blood vessels and releases hormones such as aldosterone and norepinephrine, by inhibiting an enzyme called angiotensin converting enzyme. Angiotensin II, aldosterone, and norepinephrine all increase blood pressure and urine production by the kidneys. If levels of these three substances decrease in the body, this allows blood vessels to relax and dilate (widen), reducing both blood and kidney pressure. Angiotensin-converting enzyme (ACE) inhibitors include, but are not limited to, benazepril (LOTENSIN), captopril, enalapril (VASOTEC), fosinopril, lisinopril (PRINIVIL and ZESTRIL), moexipril, perindopril, quinapril (ACCUPRIL), ramipril (ALTACE), and trandolapril

Angiotensin II receptor blockers (ARBs) have similar effects as ACE inhibitors, but work by a different mechanism. These drugs block the effect of angiotensin II, a chemical that narrows blood vessels. By doing so, they help widen blood vessels to allow blood to flow more easily, which lowers blood pressure. Examples of ARBs include, but are not limited to ATACAND (candesartan), AVAPRO (irbesartan), BENICAR (olmesartan), COZAR (losartan), DIOVAN (valsartan), MICARIS (telmisartan), and TEVETAN (eprosartan).

In some applications, a method of the present disclosure may be used for the presymptomatic treatment of individuals, beginning after the determination or diagnosis of Alport syndrome and prior to the onset of symptoms, such as for, example, proteinuria. The diagnosis of Alport syndrome in an individual may be made, for example, by family medical history, genetic testing, immunodiagnostic skin biopsy testing, and/or molecular diagnostic marker testing. Methods of the present disclosure may also include one or more steps of obtaining a diagnosis of Alport syndrome by the use of one or more such diagnostic means.

The agents of the present methods may be administered separately or as part of a mixture of cocktail. With the present methods, one agent may be administered before, after, and/or coincident to with the administration of a second agent.

The agents of the present methods may be administered at once or may be divided into a number of smaller doses to be administered at intervals of time. For example, agents may be administered twice a day, three times a day, four times a day, or more. For example, agents may be administered every other day, every third day, once a week, every two weeks, or once a month. In some applications, agents may be administered continuously, for example by a controlled release formulation or a pump. In some applications, administration on antibody of the present disclosure may be at a dosage similar to the accepted dosage for other therapeutic antibodies.

The agents of the present methods may be administered by any suitable means including, but not limited to, for example, oral, rectal, nasal, topical (including transdermal, aerosol, buccal and sublingual), vaginal, parenteral (including subcutaneous, intramuscular, intravenous and intradermal), intravesical, or injection into or around the tumor. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intraperitoneal, and intratumoral administration. In this connection, sterile aqueous media that can be employed will be known to those of skill in the art. Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by the FDA. Such preparation may be pyrogen-free.

For enteral administration, an agent may be administered in a tablet or capsule, which may be enteric coated, or in a formulation for controlled or sustained release. Many suitable formulations are known, including polymeric or protein microparticles encapsulating drug to be released, ointments, gels, or solutions which can be used topically or locally to administer drug, and even patches, which provide controlled release over a prolonged period of time. These can also take the form of implants.

The present disclosure includes compositions of that include both an α1 integrin blocking agent and either an angiotensin-converting enzyme (ACE) inhibitor or an angiotensin-receptor blocker (ARB) to the subject. The al integrin blocking agent may be an α1 integrin neutralizing antibody. The al integrin blocking agent may be a small molecule inhibitor, such as, for example, obtustatin. The ACE inhibitor may, for example, be selected from benazepril, captopril, enalapril, fosinopril, lisinopril, moexipril, perindopril, quinapril, ramipril, and/or trandolapril. The ARB may, for example, be selected from candesartan, irbesartan, olmesartan, losartan, valsartan, telmisartan, and/or eprosartan. A composition may also include, for example, buffering agents to help to maintain the pH in an acceptable range or preservatives to retard microbial growth. Such compositions may also include a pharmaceutically acceptable carrier. As used herein, the term “pharmaceutically acceptable carrier” refers to one or more compatible solid or liquid filler, diluents or encapsulating substances which are suitable for administration to a human or other vertebrate animal. The compositions of the present disclosure are formulated in pharmaceutical preparations in a variety of forms adapted to the chosen route of administration.

With the present methods, agent(s) may be administered at once, or may be divided into a number of smaller doses to be administered at intervals of time. For example, an agent may be administered twice a day, three times a day, four times a day, or more. For example, an agent may be administered every other day, every third day, once a week, every two weeks, or once a month at once, or may be divided into a number of smaller doses to be administered at intervals of time. In some applications, an agent may be administered continuously, for example by a controlled release formulation or a pump.

It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions and methods.

Therapeutically effective concentrations and amounts may be determined for each application herein empirically in known in vitro and in vivo systems, such as those described herein, dosages for humans or other animals may then be extrapolated therefrom. With the methods of the present disclosure, the efficacy of the administration of one or more agents may be assessed by any of a variety of parameters known in the art.

In some therapeutic embodiments, an “effective amount” of an agent is an amount that results in a reduction of at least one pathological parameter. Thus, for example, in some aspects of the present disclosure, an effective amount is an amount that is effective to achieve a reduction of at least about 10%, at least about 15%, at least about 20%, or at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, compared to the expected reduction in the parameter in an individual not treated with the agent.

The inventions defined in the claims. However, below is provided a non-exhaustive list of non-limiting embodiments. Any one or more of the features of these embodiments may be combined with any one or more features of another example, embodiment, or aspect described herein.
1. A method of treating Alport syndrome in a subject, the method comprising administering both an α1 integrin blocking agent and an angiotensin-converting enzyme (ACE) inhibitor or an angiotensin-receptor blocker (ARB) to the subject.
2. A method of preventing glomerular disease progression in a subject diagnosed with Alport syndrome, the method comprising administering both an α1 integrin blocking agent and an angiotensin-converting enzyme (ACE) inhibitor or an angiotensin-receptor blocker (ARB) to the subject.
3. A method of treating glomerulonephritis in a subject, the method comprising administering both an α1 integrin blocking agent and an angiotensin-converting enzyme (ACE) inhibitor or an angiotensin-receptor blocker (ARB) to the subject.
4. A method of treating glomerular injury due to biomechanical strain in Alport syndrome, the method comprising administering both an α1 integrin blocking agent and an angiotensin-converting enzyme (ACE) inhibitor or an angiotensin-receptor blocker (ARB) to the subject.
5. A method of inhibiting deposition of laminin 211 in the glomerular basement membrane (GBM) in a subject, the method comprising administering both an α1 integrin blocking agent and an angiotensin-converting enzyme (ACE) inhibitor or an angiotensin-receptor blocker (ARB) to the subject.
6. A method of inhibiting mesangial cell process invasion of the glomerular capillary loop in a kidney of a subject, the method comprising administering both an α1 integrin blocking agent and an angiotensin-converting enzyme (ACE) inhibitor or an angiotensin-receptor blocker (ARB) to the subject.
7. A method of inhibiting Alport glomerular pathogenesis in a subject; the method comprising:
   determining that the subject is at risk for developing Alport glomerular disease; and
   administering both an α1 integrin blocking agent and an angiotensin-converting enzyme (ACE) inhibitor or an angiotensin-receptor blocker (ARB) to the subject.
8. The method of embodiment 7, wherein the determination that the subject is at risk for developing Alport glomerular disease is determined by family medical history, genetic testing, immunodiagnostic skin biopsy testing, and/or molecular diagnostic marker testing.
9. The method of embodiment 7 or 8, wherein the determination that the subject is at risk for developing Alport glomerular disease is made prior to the onset of proteinuria in the subject.
10. The method of any one of embodiments 1 to 9, wherein one or more sensory and/or hearing losses associated with Alport syndrome is treated.
11. The method of any one of embodiments 1 to 10, wherein the al integrin blocking agent comprises an α1 integrin neutralizing antibody.
12. The method of any one of embodiments 1 to 10, wherein the al integrin blocking agent comprises a small molecule inhibitor.
13. The method of embodiment 12, wherein the small molecule inhibitor comprises obtustatin.
14. The method of any one of embodiments 1 to 13, wherein the al integrin blocking agent prevents signaling through the α1β1 integrin receptor.
15. The method of any one of embodiments 1 to 14, wherein the ACE inhibitor is selected from benazepril, captopril, enalapril, fosinopril, lisinopril, moexipril, perindopril, quinapril, ramipril, and/or trandolapril.
16. The method of any one of embodiments 1 to 14, wherein the ACE inhibitor is selected from ramipril and/or analapril.
17. The method of any one of embodiments 1 to 14, wherein the ARB is selected from candesartan, irbesartan, olmesartan, losartan, valsartan, telmisartan, and/or eprosartan.
18. The method of any one of embodiments 1 to 17, wherein the administration of both an α1 integrin blocking agent and either an ACE inhibitor or an ARB is synergistic in protecting renal function in Alport syndrome.

The present disclosure is illustrated by the following examples. It is to be understood that the particular examples, materials, amounts, and procedures are to be interpreted broadly in accordance with the scope and spirit of the inventions as set forth herein.

### EXAMPLES

### Example 1

### Combination Therapy for Alport Renal Disease

We have previously shown that deletion of α1β1 integrin in autosomal Alport mice, which is expressed on mesangial cells, attenuates glomerular disease and results in a significant increase in lifespan (Cosgrove et al., 2000, Am J Pathol; 157(5):1649-1659). More recently we showed that α1β1 deletion in the Alport mouse markedly slowed the invasion of glomerular capillaries by mesangial filopodia, and thus prevented the deposition of laminin 211 (and presumably collagen 3 α1) in the GBM (Zallocchi et al., 2013, Am J Pathol; 183(4):1269-1280 (doi: 10.1016/j.ajpath.2013.06.015).

This example shows that both laminin 211 and collagen 3 α1 deposition are attenuated in the α1-null Allport mouse, and the deposition of these ECM molecules can be further delayed by administration of ramipril. Importantly, ramipril administration in α1-null Alport mice provided synergistic improvement of renal health and lifespan, increasing lifespan of the autosomal Alport mice to >30 weeks, which is nearly double that of al-null Alport mice (mean lifespan of 16 weeks), and triple that of Alport mice (mean lifespan of 10 weeks). This increase in lifespan is unprecedented in the field. Importantly, this is the first example where a potential targeted treatment shows synergistic benefit when combined with ACE inhibitors, which are the current standard of care. It is notable that humanized al integrin neutralizing antibodies exist and have made it through phase II clinical trials. These antibodies have been shown to functionally inhibit inflammatory disease as well as the al-null mutation in mice (de Fougerolles et al., 2000, J Clin Invest; 105(6):721-729; Fiorucci et al., 2002, Immunity; 17(6):769-780 (doi: 10.1016/s1074-7613(02)00476-4); and Conrad et al., 2007, Nature Medicine; 13(7):836-842 (doi: 10.1038/nm1605)).

This example provides a new use for these antibodies in a dual therapy where both ramipril treatment and al integrin blockade are employed to prevent mesangial filopodial invasion and the deposition of mesangial proteins in the GBM of Alport patients, resulting in improved renal function, slowed progression of proteinuria and increase in lifespan over ramipril therapy alone.

Twenty years ago, we published a paper that described how the deletion of the mesangial cell integrin, α1β1, in the Alport mouse model resulted in attenuated pathology and a 50% increase in lifespan (Cosgrove et al., 2000, Am J Pathol; 157(5):1649-1659). In this same paper we pointed out for the first time that laminin α2 was accumulating in the basement membranes and the accumulation was attenuated in the al integrin null Alport mice at 7 weeks of age. The source of laminin 211, mesangial filopodia that invade the subendothelial aspect of the glomerular capillaries, was not identified until much later (Zallocchi et al., 2013, Am J Pathol; 183(4):1269-1280 (doi: 10.1016/j.ajpath.2013.06.015)). The fact that laminin 211 was directly injuring podocytes and contributing to the pathobiology of glomerular disease was shown even later (Delimont et al., 2014, PLoS ONE; 9(6) (doi: 10.1371/journal.pone.0099083)). A number of therapies are moving into human clinical trials including Lademirsen (SAR339375), which is a modified anti-mir21 and showed reasonable efficacy in pre-clinical work using Alport mice with a 30% increase in lifespan (Gomez et al., J Clin Invest; 125(1):141-156 (doi: 10.1172/JCI75852)), and Bardoxolone methyl, which has not been tested in the mouse models.

However, no therapeutic intervention to date has been shown to provide significant improvement of renal function and lifespan over ACE inhibitors (typically ramipril) or ARBs. ACE blockers or ARBs are the current standard of care for Alport patients. ACE inhibitors have been shown in retrospective studies to significantly increase lifespans of patients with Alport syndrome (Gross et al., 2012, ISRN Pediatr; 2012:436046 (doi: 10.5402/2012/436046); Gross et al., 2012, Kidney Int; 81(5):494-501 (doi: 10.1038/ki.2011.407); Gross et al., 2020, Kidney Int; 97(6):1275-1286 (doi: 10.1016/j.kint.2019.12.015); and Rheault and Smoyer, 2020, Kidney Int; 97(6):1104-1106 (doi: 10.1016/j.kint.2020.01.030)), and thus any therapy that is to be adopted in the field must show significant benefit over ACE or ARBs alone. To date, no therapeutic has been shown to have this property.

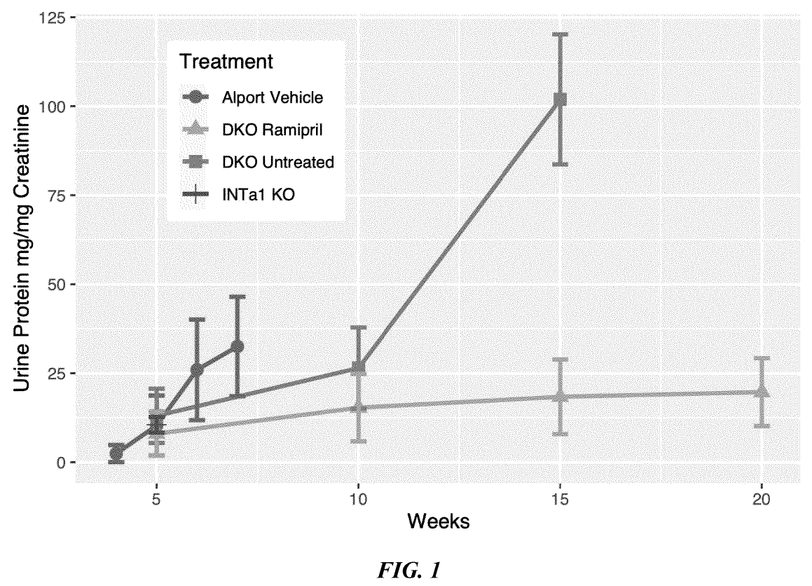

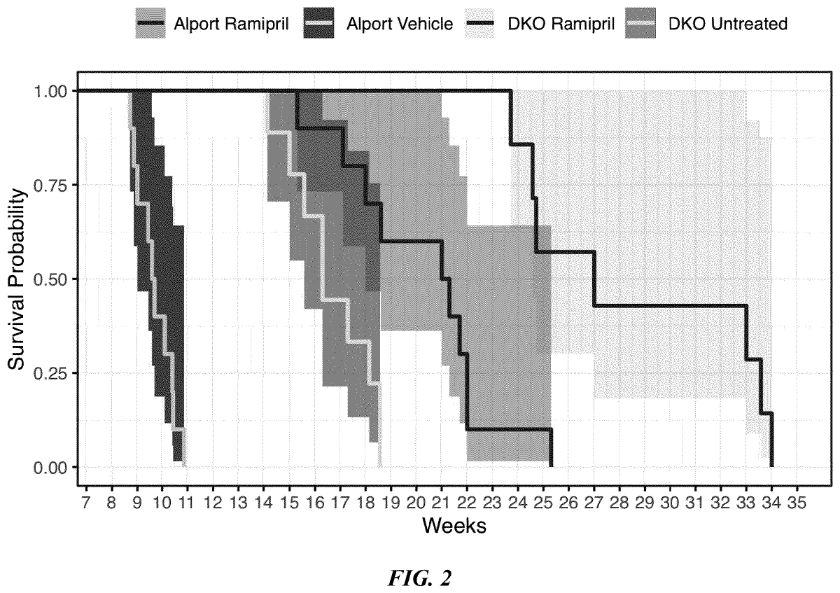

Autosomal Alport mice or al integrin null Alport (DKO) mice (both on the 129 Sv background) were given straight water or treated with ramipril (10 mg/kg/day in drinking water) starting at 3 weeks of age. Proteinuria (FIG. 1) and lifespan (FIG. 2) were measured. We were surprised to see the improvement of lifespan from 20 weeks in ramipril-treated Alport mice to >30 weeks in ramipril-treated DKO mice. Tripling the lifespan of the Alport mouse model (which lives to on average 10 weeks of age as is evident in FIG. 2) is unprecedented in the Alport field. Integrin α1-null Alport (DKO) mice treated with ramipril live twice as long as untreated DKO mice and 3 times as long as untreated Alport mice. Animals from the indicated treatment groups were allowed to live until they lost >10% of their peak body weight and then euthanized (a humane endpoint, considered age of death). DKO mice show a 60% increase in lifespan. Thus, this tripling of lifespan shows the integrin α1 blockade provides synergistic improvement in lifespan over ramipril treatment alone. If translated to humans (as did ramipril treatment starting at a young age) this therapy could portend survival to 60-70 years of age for Alport patients with severe mutations.

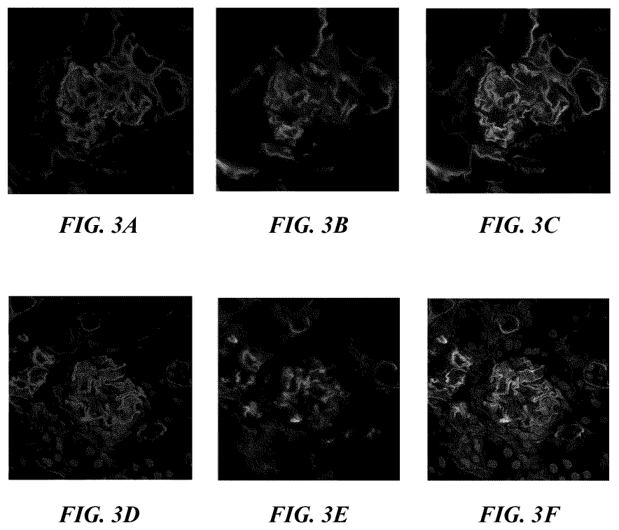

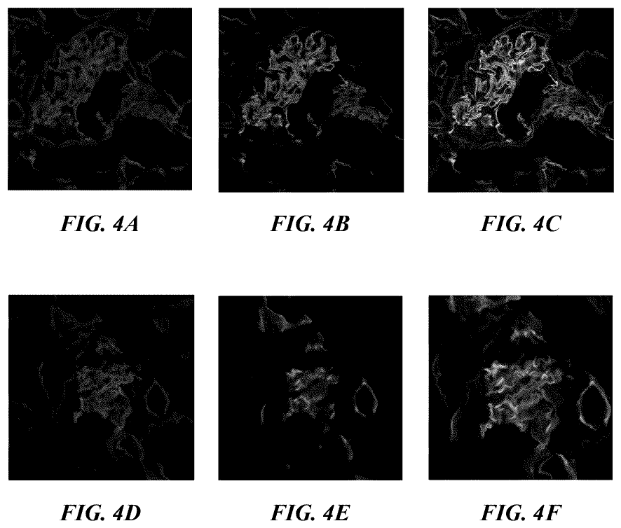

The status of mesangial filopodial invasion was examined at these timepoints and found that, in the Ramipril-treated DKO mice there was no laminin 211 or collagen III in the GBM at 10 weeks of age, while in the untreated DKO mice there was a significant amount of these proteins present (FIG. 3 and FIG. 4). This evidence has obvious and important translational value. By preventing the invasion of the glomerular capillaries by mesangial filopodia, the deposition of mesangial proteins in the GBM is prevented. This prevents podocyte injury that is caused by the mesangial matrix.

This presents an opportunity to gain additional insight into the role of biomechanical strain on the transcriptome in Alport mice minus the confounding effects of mesangial ECM protein-mediated podocyte injury. We noted in earlier work that biomechanical strain accelerated the progression of Alport glomerular disease, including GBM damage and deposition of laminin 211 in the GBM (Meehan et al., 2009, Kidney Int; 76(9):968-976 (doi: 10.1038/ki.2009.324)). The reduction of blood pressure by ramipril and ARBs likely contributes significantly to the renoprotective effects of this preemptive therapy.

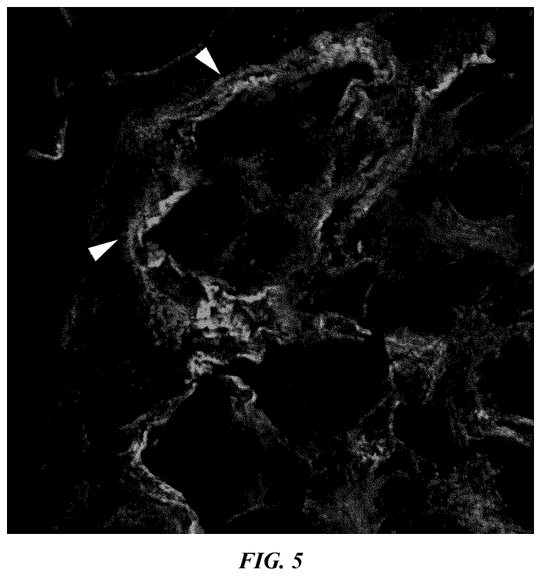

We know mesangial filopodial-derived laminin α2 plays a role in podocyte injury in Alport syndrome (Delimont et al., 2014, PLoS ONE; 9(6) (doi: 10.1371/journal.pone.0099083)). There are two collagen receptors expressed on glomerular podocytes; integrin α2B1 and DDR1. Deletion of either of these two receptors in Alport mice results in attenuated progression of renal disease and extended lifespan, clearly implicating collagen mediated signaling via these receptors in the pathobiologic mechanism of Alport glomerular disease in the model (Gross et al., 2010, Matrix Biol; 29(5):346-56; and Rubel et al., 2014, Matrix Biol; 34:13-21 (doi: 10.1016/j.matbio.2014.01.006)). Super resolution microscopy studies suggested that the type IV collagen α3/α4/α5 network was too distant from the podocyte pedicles to interact with collagen receptors, while in Alport mice the resulting type IV collagen α1/α2 network was sufficiently proximal to podocyte pedicles to interact (Suleiman et al., 2013, ELife; 2:e01149 (doi: 10.7554/eLife.01149)). There is no direct evidence that this in fact occurs or that collagen IV network activates collagen receptors. We performed dual immunofluorescence super resolution structure illumination microscopy (SR-SIM) using antibodies against collagen III (green) and α-actinin 4, which localizes to the podocyte foot processes (red), to determine whether collagen III in the GBM of Alport mice was proximal to the podocyte foot processes. The results in FIG. 5 show that this is indeed the case and thus collagen III may indeed activate collagen receptors on glomerular podocytes, resulting in podocyte injury.

### Example 2

### Evaluating the Role of ECM Deposition in the GBM as a Contributor to Glomerular Pathology

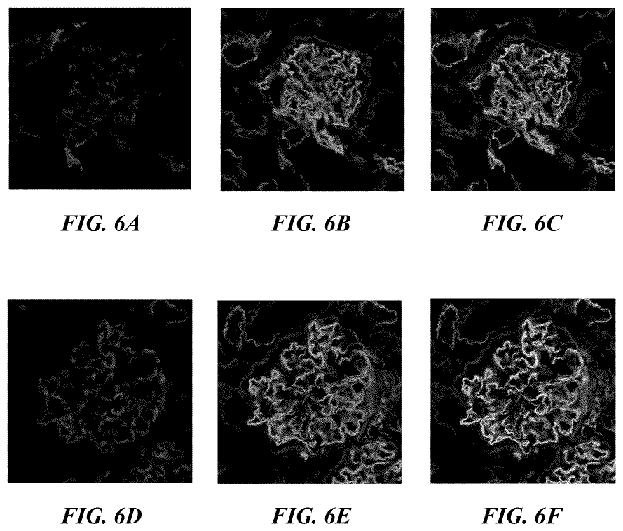

If the ECM deposition in the GBM is indeed a significant contributor to glomerular pathology we would expect it would be delayed in the ramipril-treated DKO mice. To determine if this was indeed the case, we performed immunohistochemical analysis for laminin 211 dual stained with the GBM marker laminin α5. Specifically, Duan immunofluorescence analysis was performed on cryosections from 20 and 25 week old DKO/ramipril mice. Sections were immunostained using antibodies for laminin 211 and laminin α5 (a GBM marker). We did not observe any significant GBM accumulation before 20 weeks of age. Between 20 and 25 weeks of age, however, GBM localization of laminin 211 became widespread as depicted in FIGS. 6A-6F. FIGS. 6A-6C are the 20-week data and FIGS. 6D-6F are the 25-week data. These figures show that Laminin 211 is deposited in the GBM in ramipril-treated DKO mice after 20 weeks and at or before 25 weeks of age.

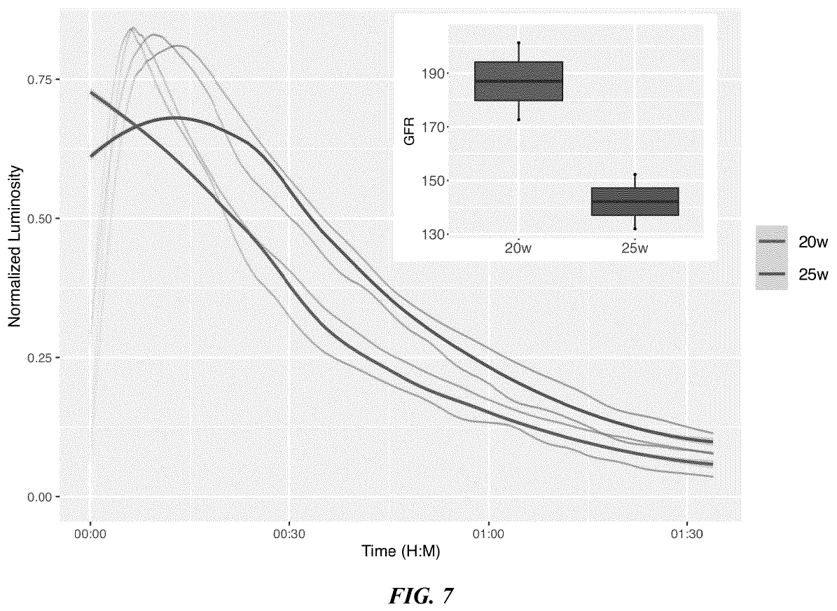

Glomerular filtration rates (GFR) were measured using the MediBeacon (St Louis, MO) transdermal LED approach, which allows serial measurements to be conducted in the same animals. The results in FIG. 7 show that GFR is reduced in DKO ramipril mice at 25 versus 20 weeks of age. A marked reduction in GFR is observed at 25 weeks relative to 20 weeks in ramipril-treated DKO mice. Serial GFR measures were made using two ramipril-treated DKO mice. GFR measures were at 20 and 25 weeks of age. Additional mice at additional timepoints of measure (to 30 weeks) will be added as part of the research plan to establish statistical significance.

Given the abundance of GBM laminin 211 in the 25-week versus 20-week-old DKO ramipril mice, we surmised that this could provide an in vivo model to understand laminin 211-mediated pathology. To this end we performed RNA-seq analysis on glomeruli from 20 and 25-week-old DKO ramipril mice (3 animals per time-point). The data was analyzed as follows: Read counts were calculated utilizing the Trimmomatic suite (Bolger et al., 2014) and a 2 pass STAR run (Dobin et al., 2013) with Rsubread (Liao et al., 2019) as the constituent read calculator. Mm10 was the reference genome used, with a GTF file pruned to matching human orthologs, and also genes concurrent with the Mouse Genome Index. PGK1, and GAPDH were used for the normalizing factors (Panina et al., 2018).

The resulting normalized counts were input into the Gene Set Enrichment Analysis from the Broad Institute (Subramanian et al.), to determine differential expression. We performed this analysis utilizing GSEA's signal to noise ratio metric, to allow us to find induction or suppression, from or to zero read count values for each treatment vs. control. We utilized the rank ordered gene list from GSEA's output to determine lists of differentially expressed genes for each. The results aligned with the cell culture data for laminin 211-treated versus non-treated podocytes. The results are summarized in Table 1, where the Lam 211 FC numbers represent fold change based on total normalized read counts. The Accession Numbers are provided for each gene so that information including, but not limited to the genetic sequence, protein sequence, and transcript sequence can be accessed. These sequences should be considered incorporated fully herein in their entirety.

TABLE 1

| | | Encoded protein | | Lam | DKO |
|---|---|---|---|---|---|
| Gene | Accession No. | (protein function in kidney) | Reference | 211 FC | FC |
| AGT | NG_008836 | Angiotensinogen (podocyte injury) | Koizumi et al. | 2.71 | 1.49 |
| AQP11 | NM_173039.3 | Aquaporin 11 (CKD) | Tanaka et al. | 2.38 | 1.57 |
| AQP7 | NG_027764 | Aquaporin 7 (CKD) | Noda et al. | 2.38 | 3.16 |
| C3 | NG_009557 | Complement Component 3 (podocyte injury) | Gao et al. | 7.31 | 1.47 |
| CFB | NG_008191 | Complement Factor B (CKD) | Lemaire Li et al. | 2.52 | 1.27 |
| F5 | NG_011806 | Coagulation Factor V (CKD) | Yang et al. | 2.86 | 1.83 |
| FGF10 | NG_011446 | Fibroblast Growth Factor 10 (nephroprotection) | Tan et al. | 2.69 | 10.51 |
| GATM | NG_011674 | Glycine Amidinotransferase (CKD) | Reichold et al. | 2.38 | 4.01 |
| GC | NG_012837 | Vitamin D Binding Protein (podocyte injury) | Gembillo et al. | 3.46 | 18.90 |
| IHH | NG_016741 | Indian Hedgehog (CKD) | Maghmomeh et al. | 5.72 | 2.41 |
| NOTUM | NP_848588.3 | Notum Palmitoleoyl-Protein Carboxylesterase (kidney devel.) | Vogel et al. | 6.20 | 1.65 |
| SLC1A1 | NG_017044 | Solute Carrier Family 1 (Neuronal/Epithelial High Affinity | Bailey et al. | 2.17 | 1.90 |
| | | Glutamate Transporter, System Xag), Member 1 (CKD) | | | |
| SLC22A13 | NM_004256 | Solute Carrier Family 22 (Organic Cation Transporter), | Nigam et al. | 4.77 | 2.75 |
| | | Member 13 (CKD) | | | |
| SLC5A11 | NM_001352248.3 | Solute Carrier Family 5 (Sodium/Glucose Cotransporter), | Gil et al. | 2.44 | 4.40 |
| | | Member 11 (CKD) | | | |
| ADGRL3 | NG_033950 | Adhesion G Protein-Coupled Receptor L3 (podocyte injury) | Cazorla-Vázquez et al. | −2.10 | −4.49 |
| BMP2 | NG_023233 | Bone Morphogenetic Protein 2 (CKD) | Pache et al. | −2.10 | −3.81 |
| BMP7 | NG_032771 | Bone Morphogenetic Protein 7 (nephroprotection) | Ohigashi et al. | −5.77 | −4.29 |
| EGR3 | NM_004430.3 | Early Growth Response 3 (inflammation) | Wieland et al. | −1.92 | −5.34 |
| FOXC2 | NG_012025 | Forkhead Box C2 (nephroprotection) | Nilsson et al. | −2.40 | −4.29 |
| GCK | NG_008847 | Glucokinase (nephroprotection) | Yang et al. | −1.95 | −4.90 |
| NEBL | NG_017092 | Nebulette (nephroprotection) | Ge et al. | −2.10 | −4.22 |
| NPHS1 | NG_013356 | Nephrosis 1, Nephrin (podocyte injury) | Fukusumi et al. | −2.10 | −5.64 |
| NPHS2 | NG_007535 | Nephrosis 2, Podocin (podocyte injury) | Zhang et al. | −2.10 | −4.10 |
| NR4A3 | NG_028910 | Nuclear Receptor Subfamily 4, Group A, Member 3 | Shi et al. | −2.10 | −8.38 |
| | | (nephroprotection) | | | |
| ROBO2 | NG_027734 | Roundabout Guidance Receptor 2 (nephroprotection) | Pisarek-Horowitz et al. | −2.50 | −4.21 |

While all the genes listed in Table 1 have been previously implicated in podocyte injury, protection or biology, of particular interest is the fact that podocin and nephrin mRNAs are both significantly down regulated in both cell culture experiments and in vivo. A recent paper (Yang et al., JASN 32: 1323-1337, 2021) showed that the transcription factor FOXC2 binds a super enhancer to regulate NEPHS1 (encoding nephrin) and NEPHS2 (encoding podocin) gene expression. FOXC2 expression is also down regulated in laminin 211-treated cells and in vivo. This remarkable finding may constitute a key mechanism underlying Alport glomerular pathology.

### Example 3

### ROLE OF α1β1 GLOMERRULAR PATHOGENESIS

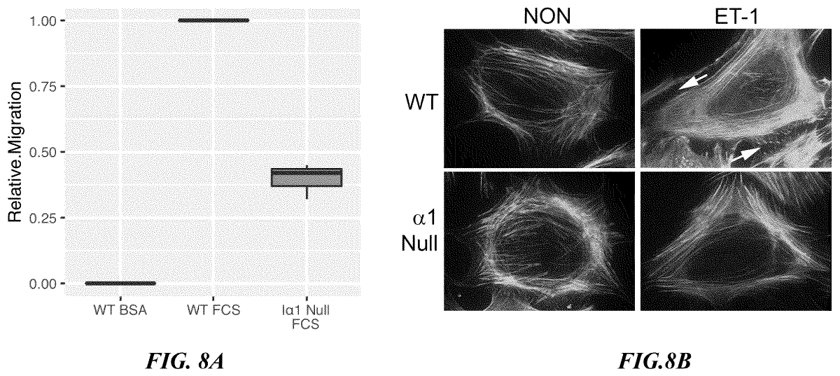

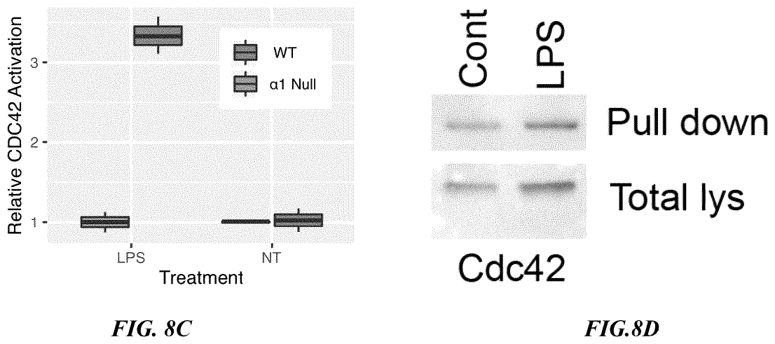

Our earlier studies revealed that integrin alol played a role in Alport glomerular pathogenesis (Cosgrove et al., 2000), however, the mechanism was never clear. We showed that endothelin-1 activation of ET_{A}R receptors play a key role in activating mesangial filopodial invasion of the GBM and deposition of laminin 211 via crosstalk between Rac1 and CDC42 (Zallocchi et al., 2013). By extrapolation, it seemed logical that alol integrin may function along the CDC42 activation axis in cultured mesangial cells. To test this, we performed several assays whose data is provided in FIGS. 8A-8D.

FIG. 8A shows the results of a Boyden chamber cell migration assay. The results show that al-null mesangial cells migrate much less than wild type mesangial cells in response to fetal calf serum. FIG. 8B shows that treatment of wild type mesangial cells with endothelin-1 activates the formation of filopodia (arrows) while al-null mesangial cells do not respond to ET-1 treatment. FIG. 8C shows the results of an ELISA assay for activated CDC42. Treatment of wild type mesangial cells with lipopolysaccharide (LPS) activates CDC42, while LPS treatment of al-null cells does not. Lastly, FIG. 8D shows that LPS did not activate CDC42 over untreated cells as demonstrated in pull-down assays. Thus, it appears that the al-null mesangial cells are refractive to activation of CDC42, which is consistent with the markedly attenuated effect on mesangial filopodial invasion, explaining why al integrin blockade attenuates ECM-mediated podocyte injury. In summary, these assays and the data provided in FIGS. 8A-8D demonstrate that al-null mesangial cells show reduced migration, failure to form filopodia in response to ET-1 stimulation, and failure to activate CDC42 in response to LPS.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The inventions are not limited to the exact details shown and described, for variations obvious to one skilled in the art will be included within the inventions defined by the claims.

What is claimed is:

1. A method of treating Alport syndrome in a subject, the method comprising administering both an α1 integrin blocking agent and an angiotensin-converting enzyme (ACE) inhibitor or an angiotensin-receptor blocker (ARB) to the subject.

2. A method of preventing glomerular disease progression in a subject diagnosed with Alport syndrome, the method comprising administering both an α1 integrin blocking agent and an angiotensin-converting enzyme (ACE) inhibitor or an angiotensin-receptor blocker (ARB) to the subject.

3. A method of treating glomerulonephritis in a subject, the method comprising administering both an α1 integrin blocking agent and an angiotensin-converting enzyme (ACE) inhibitor or an angiotensin-receptor blocker (ARB) to the subject.

4. A method of treating glomerular injury due to biomechanical strain in Alport syndrome, the method comprising administering both an α1 integrin blocking agent and an angiotensin-converting enzyme (ACE) inhibitor or an angiotensin-receptor blocker (ARB) to the subject.

5. A method of inhibiting deposition of laminin 211 in the glomerular basement membrane (GBM) in a subject, the method comprising administering both an α1 integrin blocking agent and an angiotensin-converting enzyme (ACE) inhibitor or an angiotensin-receptor blocker (ARB) to the subject.

6. A method of inhibiting mesangial cell process invasion of the glomerular capillary loop in a kidney of a subject, the method comprising administering both an α1 integrin blocking agent and an angiotensin-converting enzyme (ACE) inhibitor or an angiotensin-receptor blocker (ARB) to the subject.

7. A method of inhibiting Alport glomerular pathogenesis in a subject; the method comprising:
determining that the subject is at risk for developing Alport glomerular disease; and
administering both an α1 integrin blocking agent and an angiotensin-converting enzyme (ACE) inhibitor or an angiotensin-receptor blocker (ARB) to the subject.

8. The method of claim 7, wherein the determination that the subject is at risk for developing Alport glomerular disease is determined by family medical history, genetic testing, immunodiagnostic skin biopsy testing, and/or molecular diagnostic marker testing.

9. The method of claim 7 or 8, wherein the determination that the subject is at risk for developing Alport glomerular disease is made prior to the onset of proteinuria in the subject.

10. The method of any one of claims 1 to 9, wherein one or more sensory and/or hearing losses associated with Alport syndrome is delayed and/or treated.

11. The method of any one of claims 1 to 10, wherein the al integrin blocking agent comprises an α1 integrin neutralizing antibody.

12. The method of any one of claims 1 to 10, wherein the al integrin blocking agent comprises a small molecule inhibitor.

13. The method of claim 12, wherein the small molecule inhibitor comprises obtustatin.

14. The method of any one of claims 1 to 13, wherein the al integrin blocking agent prevents signaling through the α1β1 integrin receptor.

15. The method of any one of claims 1 to 14, wherein the ACE inhibitor is selected from benazepril, captopril, enalapril, fosinopril, lisinopril, moexipril, perindopril, quinapril, ramipril, and/or trandolapril.

16. The method of any one of claims 1 to 14, wherein the ACE inhibitor is selected from ramipril and/or analapril.

17. The method of any one of claims 1 to 14, wherein the ARB is selected from candesartan, irbesartan, olmesartan, losartan, valsartan, telmisartan, and/or eprosartan.

18. The method of any one of claims 1 to 17, wherein the administration of both an α1 integrin blocking agent and either an ACE inhibitor or an ARB is synergistic in protecting renal function in Alport syndrome.
